SEQUENCE LISTING (5 sequences)

SEQ ID NO: 1 - DNA, 21 bp, synthetic construct
  misc_feature 1..21: note = Synthetic
  source 1..21: mol_type = other DNA
acgcctgtaatcccagcactt

SEQ ID NO: 2 - DNA, 19 bp, synthetic construct
  misc_feature 1..19: note = Synthetic
  source 1..19: mol_type = other DNA
tcgcccaggctggagtgca

SEQ ID NO: 3 - DNA, 21 bp, synthetic construct
  misc_feature 1..21: note = Synthetic
  source 1..21: mol_type = other DNA
aggtcggtgtgaacggatttg

SEQ ID NO: 4 - DNA, 19 bp, synthetic construct
  misc_feature 1..19: note = Synthetic
  source 1..19: mol_type = other DNA
ggggtcgttgatggcaaca

SEQ ID NO: 5 - protein, 28 aa, synthetic construct
  REGION 1..28: note = Synthetic
LSTAADMQGVVTDGMASGLDKDYLKPDD

(19) United States
(12) Patent Application Publication
(10) Pub. No.: US 2025/0302306 A1
(43) Pub. Date: Oct. 2, 2025

(54) TUMOR TARGETING PROBE FOR IMAGE GUIDED SURGICAL METHODS

(71) Applicant: The Board of Trustees of the Universities of Illinois, Urbana, IL (US)

(72) Inventors: Tapas K. Das Gupta, River Forest, IL (US); Tohru Yamada, Oak Park, IL (US); Masahide Goto, Urbana, IL (US); Samer Naffouje, Urbana, IL (US)

(21) Appl. No.: 18/714,803

(22) PCT Filed: Nov. 30, 2022
(86) PCT No.: PCT/US2022/080631

§ 371 Date: May 30, 2024

Related U.S. Application Data
(60) Provisional application No. 63/264,732, filed on Dec. 1, 2021.

(51) Int. Cl. A61B 5/00; A61B 90/00; A61K 49/00

(52) U.S. Cl. CPC A61B 5/0071; A61B 5/0077; A61B 5/4312; A61K 49/0034; A61K 49/0056; A61B 2090/3933; A61B 2090/3941; A61B 2505/05

(57) ABSTRACT

A unique near-infrared (NIR) fluorescence imaging probe, ICG-p28, composed of the clinically nontoxic tumor-targeting peptide p28 and the FDA-approved NIR dye indocyanine green (ICG) have been analyzed for in vivo kinetics and optimized for clinical practice. Intraoperative imaging with ICG-p28 was used to identify small (≤1 mm) lymph nodes (LNs) containing cancer cells. Image-guided surgery with ICG-p28 showed an over 6.6×10³-fold reduction in residual normalized tumor DNA at the margin site relative to control approaches, resulting in an improved tumor recurrence rate (92% specificity) in multiple animal models.

### REFERENCE TO RELATED APPLICATIONS

This application claims the benefit of U.S. Provisional Application No. 63/264,732, filed Dec. 1, 2021, which application is incorporated herein by reference in its entirety.

### STATEMENT AS TO FEDERALLY SPONSORED RESEARCH

This invention was made with Government support under Federal Grant Nos. R01EB023924 awarded by the NIH. The Government has certain rights to this invention.

### REFERENCE TO A SEQUENCE LISTING

The instant application contains a Sequence Listing which has been filed electronically in ASCII format and is hereby incorporated by reference in its entirety. Said ASCII copy, created on Nov. 29, 2022, is named txt_46466-57.txt and is 2229 bytes in size.

### BACKGROUND OF THE INVENTION

Breast cancer is primarily treated via surgical excision of the tumor with the surrounding normal breast tissue margin. In the early stage, these treatments take the form of breast-conserving surgery (BCS) or lumpectomy. Although BCS provides satisfactory cosmetic outcomes, the re-resection rate is ˜20%, and of these surgeries, ˜85% are performed due to the presence of initial positive margins. Although there is often a need for further surgery for early-stage breast cancer patients and additional tissue removal owing to the inherent challenges in achieving negative margins, existing guidelines recommend against excising a wider negative margin in routine practice. Currently, surgeons rely primarily on visual and tactile cues to distinguish between healthy and malignant tissues and thus may leave residual lesions in the tumor bed. CT, MRI, PET, etc. are readily available imaging modalities typically used to assess the tumor lesion prior to the surgical procedure; specifically for breast cancer, mammography along with MRI or ultrasound is the current standard of care. Although these traditional imaging modalities have been of immense help in advancing tumor detection and determining the extent of resection(s), the need for accurate intraoperative imaging remains one of the most important unmet needs in the management of breast cancer at the time of the initial BCS.

Extensive research is ongoing to identify new, rapid, and accurate intraoperative margin assessment tools, some of which are currently undergoing clinical testing. For example, among biophotonic-based techniques, image-guided surgery using fluorescence imaging offers many advantages for surgeons. Near-infrared (NIR) imaging is an emerging biomedical imaging modality for fluorescence-guided surgery because of its significant light absorption, ability to assist in real-time visualization, and lack of ionizing radiation. In vivo imaging in the NIR range (700-900 nm) is superior to that in the visible spectrum owing to its low scattering, negligible tissue autofluorescence, and relatively high tissue penetration. The first FDA-approved NIR dye, indocyanine green (ICG), has been used in clinical practice for over fifty years due to its proven safety and the feasibility of its use. However, the primary disadvantages of using ICG for diagnosis are its very short half-life (2-4 min), its ability to readily nonspecifically bind to plasma proteins, and its lack of a tumor-specific ligand-receptor interaction mechanism as a passive fluorescent dye. Thus, creating a new tumor-targeted version of ICG could be useful in the clinic.

### SUMMARY OF THE INVENTION

Current cancer treatment involves the excision of both the tumor and the surrounding normal tissue margin, followed by systemic therapy for early and locally advanced diseases. However, further surgery and tissue removal are often necessary due to the inherent challenges associated with achieving negative margins. There is an urgent need for a novel method to optimize surgical excision and confirm tumor-free edges. We developed a unique near-infrared (NIR) fluorescence imaging probe, ICG-p28, composed of the clinically nontoxic tumor-targeting peptide p28 and the FDA-approved NIR dye indocyanine green (ICG). Herein, the in vivo kinetics were analyzed to optimize settings for clinical practice, and intraoperative imaging with ICG-p28 was used to identify small (≤1 mm) lymph nodes (LNs) containing cancer cells. Xenograft tumors stably expressing iRFP as a tumor marker showed significant colocalization with ICG-p28 but not ICG alone. Image-guided surgery with ICG-p28 showed an over 6.6×10³-fold reduction in residual normalized tumor DNA at the margin site relative to control approaches (i.e., surgery with ICG or palpation/visible inspection alone), resulting in an improved tumor recurrence rate (92% specificity) in multiple breast cancer animal models independent of the receptor expression status. ICG-p28 allowed accurate identification of tumor cells in the margin to increase the likelihood of complete resection. Our simple and cost-effective approach offers a new surgical procedure that enables surgeons to intraoperatively identify tumor margins in a real-time, 3D fashion and that notably improves overall outcomes by reducing re-excision rates.

Other methods, features and/or advantages is, or will become, apparent upon examination of the following figures and detailed description. It is intended that all such additional methods, features, and advantages be included within this description and be protected by the accompanying claims.

### BRIEF DESCRIPTION OF THE SEQUENCE LISTING

SEQ ID NO: 1 is a primer specific for human-specific Alu sequence (huAlu), sense: 5′-ACG CCT GTA ATC CCA GCA CTT-3′ (SEQ ID NO: 1).

SEQ ID NO: 2 is a primer specific for human-specific Alu sequence (huAlu), antisense: 5′-TCG CCC AGG CTG GAG TGC A-3′ (SEQ ID NO: 2).

SEQ ID NO: 3 is a primer specific for the mouse GAPDH genomic DNA sequence (mGAPDH) sense: 5′-AGG TCG GTG TGA ACG GAT TTG-3′ (SEQ ID NO: 3).

SEQ ID NO: 4 is a primer specific for the mouse GAPDH genomic DNA sequence (mGAPDH) antisense: 5′-GGG GTC GTT GAT GGC AAC A-3′ (SEQ ID NO: 4).

SEQ ID NO: 5 is p28 derived from azurin of P. aeruginosa

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A-1E: FIG. 1A is a graph of NIR fluorescence for ICG-p28 as a function of concentration. FIG. 1B is a graph of signal-to-noise ratio of the NIR signal in various conditions. FIG. 1C demonstrates NIR signals as a function of size. FIG. 1D is a graph of NIR signals as a function of depth in a gelatin based phantom. FIG. 1E schematic diagram of the creation of the gelatin-based phantom.

FIG. 2A-2C are real-time imaging with ICG-p28 in xenograft mouse models. MDA-MB-231 (FIG. 2A) or IOWA-IT (FIG. 2B) breast cancer cells were injected s.c. into mice. White light photographs (upper) and snapshot NIR PDE images are shown (lower; dorsal view, greyscale at 800 nm. FIG. 2C, H&E-stained sections of the primary tumour (upper) and a sciatic LN (lower, yellow arrowhead in b) located at the superficial gluteal muscle, which was confirmed to be tumor positive (magnification 200×(inset 40×)).

FIG. 3A-3D: Colocalization of ICG-p28 and the tumor marker iRPF. FIG. 3A, ICG-p28-treated MDA-MB-231-iRFP cells were fixed in paraformaldehyde, and images were acquired under a confocal microscope. Red: tumor marker iRFP (700 nm); green: ICG-p28 (800 nm); blue: DAPI (nucleus); yellow: red+green. FIG. 3B: Athymic mice bearing MDA-MB-231-iRFP tumours were injected i.v. with either ICG-p28 or ICG alone at 0.5 mg/kg. Twenty-four hours after injection, representative ex vivo images of the tumor and surrounding soft tissues for the ICG-p28 group (top row) and the ICG alone group (bottom row) were taken with an Odyssey scanner. The dotted line in the images indicates the outer edge of the soft tissue. iRFP (red: 700 nm); ICG-p28 or ICG (green: 800 nm); and colocalization (yellow-orange). FIG. 3C, Concordance rate of the fluorescent signal in each group [the ICG-p28 group (n=10) and the ICG alone group (n=5)]; **: P<0.01. The results are presented as the mean±SD. FIG. 3D, NIR fluorescent protein iRFP (700 nm)-expressing breast cancer cells. MDA-MB-231 cells stably expressing the iRFP gene (MDA-MB-231-iRFP) were selected in the presence of puromycin, and NIR fluorescence (greyscale at 700 nm) from iRFP was confirmed with the Odyssey® scanner.

FIG. 4A-4C: FIG. 4A, schematic diagram of the intraoperative imaging performed during tumor resection in human breast cancer xenograft tumor models. FIG. 4B, are representative images of tumor resection sites at the end of the study. FIG. 4C graphically demonstrates average volumes of the recurrent tumors (four weeks after tumor resection) in each group. The results are presented as the mean±SD.

FIG. 5A-5E: Image-guided surgery of human breast cancer PDX models. TNBC PDX cells were inoculated into the fourth abdominal fat pad. Twenty-four hours after NIR agent injection (FIG. 5A, ICG-p28, FIG. 5B, ICG alone), the tumors were resected under visualization with the PDE imaging unit. White light photographs (left) and snapshot NIR PDE images are shown (right; dorsal view, greyscale at 800 nm). Yellow arrowhead: tumor. FIG. 5C, The signal-to-noise ratio in PDX-bearing mice was recorded with the PDE unit and quantitatively analyzed (N=8 per group). ***: P<0.001. The results are presented as the mean±SD. In FIG. 5D-E, TNBC PDX cells were inoculated in the fourth abdominal fat pad. Twenty-four hours after NIR agent injection (FIG. 5D, ICG-RI p28, FIG. 5E, ICG-AA3H), the tumors were resected under guidance with the PDE imaging unit. White light photographs (left) and snapshot NIR PDE images are shown (right; dorsal view, greyscale at 800 nm). Yellow arrowhead: tumor.

FIG. 6A-6E: FIG. 6A is a schematic diagram of the sites of biopsy collection from NSG mice bearing PDX tumors. FIG. 6B is a box plot shows the values of log (huAlu DNA/mGAPDH DNA), calculated using standardized amounts of huAlu DNA and mGAPDH DNA among the agents. *: P<0.05, **: P<0.005. Results are presented as the mean±SD. FIG. 6C demonstrates histological analysis of surgical specimens. Representative images (200×) of H&E staining and staining for the proliferative marker Ki-67 to confirm the presence of negative margins in specimens obtained from the ICG-p28 group (top). The specimen obtained from the ICG group (bottom) shows the presence of malignant cells. FIG. 6D is a graph of tumor volumes in each group after four weeks. FIG. 6E demonstrates histological analysis of surgical specimens. Representative images (200×) of H&E staining and staining for the proliferative marker Ki-67.

FIG. 7A-7B: FIG. 7A represents the fold increase over residual tumor DNA in the surrounding tissue from the ICG-p28 group of FIG. 6B. FIG. 7B a normality test of the distribution of huAlu/mGAPDH values.

FIG. 8A-8B: H&E-stained sections from the ICG-p28 group. FIG. 8A: the surrounding mammary tissue after tumor removal showed atypical hyperplasia (arrowhead). FIG. 8B, recurrent tumor sections were obtained 4 weeks after image-guided surgery with ICG-p28. Magnification 200×(insets 40×).

### DETAILED DESCRIPTION OF THE INVENTION

### Definitions

As used in the specification and the claims, the singular forms “a,” “an,” and “the” include plural referents unless the context clearly dictates otherwise.

As used herein, the term “cell” includes either the singular or the plural of the term. The terms “isolated”, “purified” or “biologically pure” refer to material which is substantially or essentially free from components which normally accompany material as it is found in its native state. The term “heterologous DNA,” “heterologous nucleic acid sequence,” or “exogenous” and the like as used herein refers to a nucleic acid sequence wherein at least one of the following is true: (a) the sequence of nucleic acids foreign to (i.e., not naturally found in) a given host microorganism; (b) the sequence may be naturally found in a given host microorganism, but in an unnatural (e.g., greater than expected) amount; or (c) the sequence of nucleic acids comprises two or more subsequences that are not found in the same relationship to each other in nature.

“Peptide” and “polypeptide” are used interchangeably herein and refer to a compound made up of a chain of amino acid residues linked by peptide bonds. An “active portion” of a polypeptide means a peptide that is less than the full length polypeptide, but which retains measurable biological activity and retains biological detection

As used herein, the term “tumor” refers to any neoplastic growth, proliferation or cell mass whether benign or malignant (cancerous), whether a primary site lesion or metastases

As used herein “therapeutically effective amount” refers to an amount of a composition that relieves (to some extent, as judged by a skilled medical practitioner) one or more symptoms of the disease or condition in a mammal. Additionally, by “therapeutically effective amount” of a composition is meant an amount that returns to normal, either partially or completely, physiological or biochemical parameters associated with or causative of a disease or condition. A clinician skilled in the art can determine the therapeutically effective amount of a composition in order to treat or prevent a particular disease condition, or disorder when it is administered, such as intravenously, subcutaneously, intraperitoneally, orally, or through inhalation. The precise amount of the composition required to be therapeutically effective will depend upon numerous factors, e.g., such as the specific activity of the active agent, the delivery device employed, physical characteristics of the agent, purpose for the administration, in addition to many patient specific considerations. But a determination of a therapeutically effective amount is within the skill of an ordinarily skilled clinician upon the appreciation of the disclosure set forth herein.

Treat”, “treating”, and “treatment”, etc., as used herein, refer to any action providing a benefit to a patient at risk for or afflicted with a disease, including improvement in the condition through lessening or suppression of at least one symptom, delay in progression of the disease, prevention or delay in the onset of the disease, etc. Treatment also includes partial or total destruction of the undesirable proliferating cells with minimal destructive effects on normal cells. A subject at risk is a subject who has been determined to have an above-average risk that a subject will develop cancer, which can be determined, for example, through family history or the detection of genes causing a predisposition to developing cancer

The term “subject,” as used herein, refers to a species of mammal, including, but not limited to, primates, including simians and humans, equines (e.g., horses), canines (e.g., dogs), felines, various domesticated livestock (e.g., ungulates, such as swine, pigs, goats, sheep, and the like), as well as domesticated pets and animals maintained in zoos.

Where methods and steps described herein indicate certain events occurring in certain order, those of ordinary skill in the art will recognize that the ordering of certain steps may be modified and that such modifications are in accordance with the variations of the invention. Additionally, certain steps may be performed concurrently in a parallel process when possible, as well as performed sequentially.

The meaning of abbreviations is as follows: “C” means Celsius or degrees Celsius, as is clear from its usage, “s” means second(s), “min” means minute(s), “h,” “hr,” or “hrs” means hour(s), “psi” means pounds per square inch, “nm” means nanometers, “d” means day(s), “μL” or “uL” or “ul” means microliter(s), “mL” means milliliter(s), “L” means liter(s), “mm” means millimeter(s), “nm” means nanometers, “mM” means millimolar, “μM” or “uM” means micromolar, “M” means molar, “mmol” means millimole(s), “μmol” or “uMol” means micromole(s), “g” means gram(s), “μg” or “ug” means microgram(s) and “ng” means nanogram(s), “PCR” means polymerase chain reaction, “kDa” means kilodaltons, “g” means the gravitation constant, “bp” means base pair(s), “kbp” means kilobase pair(s), “% w/v” means weight/volume percent, “% v/v” means volume/volume percent, “rpm” means revolutions per minute, “HPLC” means high performance liquid chromatography, and “GC” means gas chromatography.

### Overview

Tumor-targeted delivery of imaging agents or drugs is one of the major challenges in cancer therapy. As targeted delivery could improve the usefulness of such agents, various delivery vehicles, including small molecules, antibodies, proteins, and peptides, have been investigated. Peptides are generally more specific than small molecules due to their high complexity and are relatively inexpensive to manufacture. In particular, cell-penetrating peptides (CPPs) have been shown to be a promising agent for improving the delivery and intracellular uptake of diagnostic and therapeutic agents. They also have advantages over greater molecular weight antibodies in terms of feasibility of synthesis, derivatization flexibility, low immunogenicity, and physicochemical parameters. These characteristics suggest that the development of CPPs as carrier molecules is a promising strategy for tumor-targeted delivery.

Pseudomonas aeruginosa azurin and its derived peptide p28 preferentially enter various cancer cells and induce p53-mediated antiproliferative effects. As a single therapeutic agent, p28 (NSC745104) was tested in two phase I clinical trials and granted the FDA Orphan Drug and Rare Pediatric Disease designations due to its demonstrated preliminary efficacy without apparent adverse effects, toxicity or immunogenicity in patients with advanced solid tumors and in pediatric patients with recurrent and refractory central nervous system (CNS) tumors (NCI and Pediatric Brain Tumor Consortium). As such, p28 is a potentially ideal CPP that can serve as a tumor-targeted carrier since it preferentially penetrates cancer cells, is highly water soluble and stable, and clinically exhibits no significant adverse effects, toxicity or immunogenicity in humans. Here, we developed a new noninvasive NIR-based probe, ICG-p28, composed of clinically nontoxic p28 and FDA-approved ICG and demonstrated that image-guided surgery with ICG-p28 clearly defines margins in a real-time, 3D fashion, resulting in adequate tumor excision in clinically relevant settings. These findings hold promise for improved outcomes in BCS and can guide the development of simplified (in terms of overall procedure/operation time) and more cost-effective protocols.

ICG-p28, a new optical imaging probe, was characterized in terms of its photophysical properties in vitro, its specific uptake in tumors and its ability to precisely identify tumor margins in multiple human breast cancer mouse models. Intraoperative imaging with ICG-p28 was able to identify small (≤1 mm) LN metastases. Image-guided surgery with ICG-p28 improved positive margin identification and the tumor recurrence rate in multiple breast cancer animal models independent of the receptor expression status. In general, fluorescence imaging is a highly beneficial technique for the real-time assessment of tumor boundaries during surgical procedures and offers several advantages, such as the relatively low cost of the fluorescent dye and safety for the patient and the medical team, as non-invasive agents are used. Additionally, optical imaging with contrast enhancement can be used to visualize tumors prior to resection without changing the surgical field of view. Due to the preferential penetration of ICG-p28, substantial contrast between the tumor and surrounding normal tissues was achieved in multiple breast cancer animal models.

It is understood that “p28 probe” may refer to any p28 molecule that is detectably labeled. Preferably the p28 comprises SEQ ID NO: 5, which in amino acids 50-77 of an azurin obtained from P. aeruginosa. It is appreciated that any polypeptide that includes SEQ ID NO: 5, or is at least 96% identical to p28 is usable as a probe in the methods described herein. That is, if a single amino acid of the 27 amino acids of p28 is altered via substitution with a different amino acid, that new sequence would be 96% identical to p28. Likewise is a single amino acid is added to the p28 sequence, would result in a sequence that is 96% identical to p28. The “p28 probe” may contain conservative amino acid substitutions or non-natural amino acids as still fall within the scope of the term “p28 probe”. Similarly it is understood that “p28 probe” may be modified with any detectably labeled substance. Though in this case preferably the detectable label is any near-infrared label.

In some aspects, herein described is a composition of a detectably labeled p28 probe, the detectable label including a near-infrared fluorescent molecule. The near-infrared fluorescent molecule is indocyanine green in some aspects.

In some aspects, herein described is a method of detecting cells expressing a tumor marker. The method includes steps of exposing the cells to a detectably labeled p28 probe and performing an imaging technique to detect the probe. The method is effective in that the probe is co-localized to cells expressing a tumor marker, and thus detects cells expressing a tumor marker. In some aspects the detectable label of the p28 probe is a near-infrared fluorescent molecule and in some aspects the molecule is indocyanine green. In some aspects, the probe demonstrates a detectable fluorescent signal at a concentration between about 0.2 pM and about 20 μM.

In some aspects, herein described is a method of intraoperatively identifying tumor cells in a subject. The subject may be suspected of having a tumor or may be a subject diagnosed to have a tumor. The method includes the steps of administering to a subject a detectably labeled p28 probe and performing surgery to remove the tumor cells from the subject, In the method, the probe is localized to tumor cells. During the surgical procedure, an imaging technique that provides real time images is performed to detect cells labeled by the probe, these cells labeled by the probe are cells. In some aspects, the detectable label of the p28 probe is a near-infrared fluorescent molecule or is indocyanine green. In some aspects, the probe is administered to the subject a concentration between about 50 nM and about 250 nM. In some aspects, the probe is administered intravenously or subcutaneously. In some aspects, the time period between administration of the probe and performing the imaging technique is about 24 hours. In some aspects, the real time imaging technique delineates the margins of a tumor during the operative procedure. In some aspects, the method is performed on a subject that has been diagnosed as having breast cancer. In some aspects, the intraoperative imaging technique includes a CCD camera unit that detect 800 nm near infra-red (NIR) signals. In some aspects, the probe is localized to a tumor that is about 1 mm in size. In some aspects it have been found that the subject has a reduced recurrence of tumors after surgical removal of a tumor when compared to subjects that had surgery to remove a tumor without the additional imaging technique during the operative procedure.

In some aspects, the invention describes postoperatively evaluating a subject for remaining tumor cells, by administering to a subject that has had surgery to remove a tumor a detectably labeled p28 probe and applying to the subject an imaging technique to detect cells labeled by the probe. The imaging technique positively identifies cells expressing a tumor marker, thus identifying any remaining tumor cells.

### EXAMPLES

### Example 1: ICG-p28 Characterization In Vitro and Kinetics In Vivo

Because ICG-p28 is a new NIR compound we characterized its photochemical properties with a PDE Neo® imaging unit, an FDA 510(k)-cleared NIR system composed of a CCD camera unit that detects 800 nm NIR signals. This PDE system (the camera coupled with a small control unit and an LCD monitor) has been used for clinical applications involving NIR imaging with ICG for identifying hepatic segments and bile ducts, among other physiological systems. The advantage of the PDE unit lies in its size and portability, which enables surgeons to hold the camera unit (1.1 lb) and easily obtain real-time NIR images from various angles during surgery. First, we tested the sensitivity of the PDE unit in detecting ICG-p28. ICG-p28 was placed in a 5% intralipid emulsion, which reportedly mimics the light scattering and absorption properties of tissue. Both ICG-p28 and ICG alone showed almost identical fluorescence quantum yields (ΦFs) at concentrations ranging from 0.2 pM to 20 μM in a concentration-dependent manner.

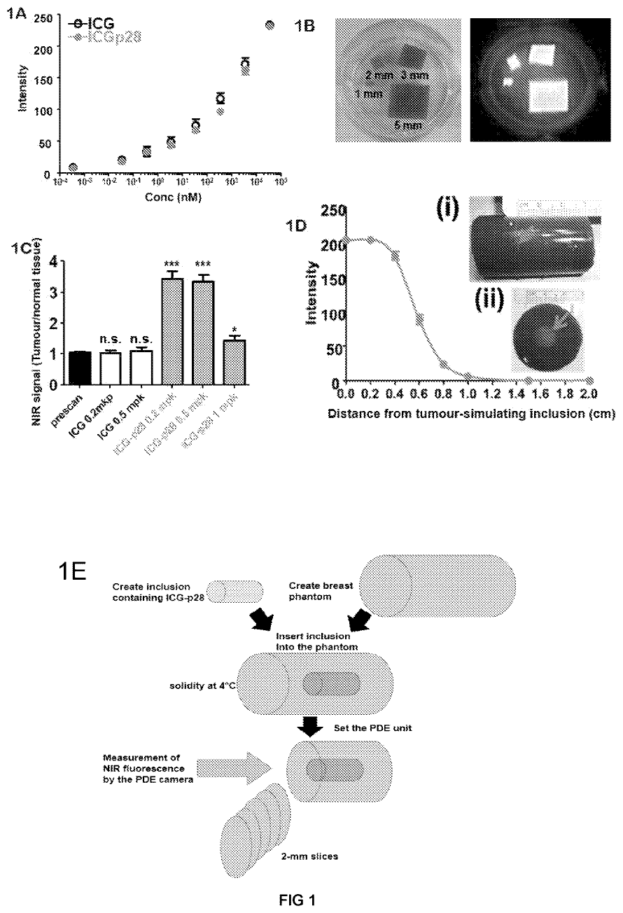

Referring now to FIG. 1E, NIR imaging of a gelatin-based breast phantom and schematic diagram of the creation of the gelatin-based phantom is described. A tube-shaped inclusion (tumor) containing 200 nM ICG-p28 in agarose was made. Subsequently, 10% melted gelatin in Tris buffer was mixed with 170 μM bovine hemoglobin and a 1% (v/v) intralipid emulsion (20%). The gelatin solution was poured into a 50-ml tube. The agarose tumor-stimulating inclusion containing ICG-p28 was placed at the center of the warm gelatin-based phantom. The phantom was allowed to solidify overnight at 4° C. Once solidified, the phantom was removed from the 50-ml tube and placed on a flat table. The PDE camera was set above the phantom (200 mm distance maintained throughout). To determine the depth detection limit, the phantom was sliced into 2-mm layers at a time until inclusion was reached. At all depths, images were taken in real time to quantitatively measure intensity.

Further referring to FIG. 1, characterization of ICG-p28 is demonstrated. FIG. 1A, NIR fluorescence from ICG-p28 or ICG in a 5% intralipid emulsion was recorded with the PDE unit at 3 mW/cm² excitation intensity (N=3). The intralipid solution alone showed no fluorescence and thus served as a control. The fluorescence quantum yield (ΦF) of ICG-p28 was similar to that of ICG at picomolar to micromolar concentrations. FIG. 1B, The signal-to-noise ratio (signal at 800 nm: tumour, noise: surrounding normal tissue, thigh muscles) in MDA-MB-231 tumour-bearing mice was recorded with the PDE unit and quantitatively analysed in a dose-dependent manner. The prescans were performed before injection of the NIR agent. P<0.001, *: P<0.05, n.s.: not significant. FIG. 1C, The specific NIR signals (800 nm) of tumour-simulating inclusions of different sizes (1, 2, 3 and 5 mm) containing 200 nM ICG-p28 were measured. FIG. 1D, The specific NIR signals (800 nm) of tumour-simulating inclusions containing 200 nM ICG-p28 inserted into phantoms at depths of 0-2 cm were measured. FIG. 1D(i), Side view of the phantom. FIG. 1D(ii), Top view of the phantom. Red arrows: locations of the tumour-simulating inclusions. The distance between the PDE camera (which captured images at 3 mW/cm² excitation intensity) and the phantom was fixed at 200 mm. The results are presented as the mean±SD.

The use of antibodies conjugated to ICG is limited, as ICG loses its NIR fluorescence due to the interaction between ICG and IgG molecules with large molecular weights/multiple domains (˜150 kDa). In contrast, when ICG was conjugated to p28, it did not lose its fluorescence, probably due to the size of p28 (2.9 kDa, ˜ 1/50th the size of IgG).

The results of preclinical and two phase I clinical trials of p28 as a monotherapeutic agent have identified the therapeutic dose of p28 for treating cancer patients. Since fluorescent imaging techniques rely on the concentration of each contrast agent, the optimum dose range for ICG-p28 in mice with MDA-MB-231 human breast cancer tumors was determined with the PDE unit. Each mouse was injected i.v. with ICG-p28 or ICG alone at 0.2-1.0 mg/kg (˜50 to 250 nM). The mice were imaged at various time points (6-72 hr), and the ratio of the NIR signal intensity of the tumor tissue and normal tissue (thigh) was calculated. Significant contrast between the tumor and surrounding normal tissues was detected at 24 hr following the injection of ICG-p28 at 0.2-1.0 mg/kg (FIG. 1B). As the higher doses of ICG-p28 (>1.0 mg/kg) required a longer time to achieve clear contrast (“washout”) because of the high background signal, the tumor-specific signal was determined to be optimal at 0.2-0.5 mg/kg concentrations within a practical time scale (24 hr) for clinical use (FIG. 1B). In contrast, ICG alone at the same dose was excreted rapidly, and the tumor site could not be distinguished (tumor/normal tissue ratio ˜1) according to the NIR images (FIG. 1B). This result illustrates the preferential uptake of p28, which leads to longer retention at the tumor site and clear contrast imaging of the tumor vs. surrounding tissue.

As described earlier, the use of optical technology at the NIR wavelength for tissue measurement is advantageous, as it can avoid the influence of autofluorescence and allow measurements of relatively deep tissues. To determine the detection limit of the objective size and depth, various sizes of tumor-simulating inclusions containing ICG-p28 (FIG. 1C) and phantoms incorporating these inclusions at predefined locations (FIG. 1D) were imaged with the PDE unit. Even with the smallest objective (1 mm), NIR fluorescence imaging of ICG-p28 at nanomolar concentrations was able to detect NIR-specific signals (FIG. 1C) and probe tissue at a depth of approximately one centimeter in an intralipid emulsion mimicking tissue scattering properties (FIG. 1D).

Based on these imaging and dosing parameters, we performed real-time PDE imaging of immunodeficient mice with two different subtypes of human breast cancer: estrogen receptor (ER)-positive breast cancer, which accounts for approximately 80% of all human breast cancers, and triple-negative breast cancer (TNBC; negative for ER, progesterone, and Her2 receptor expression), which accounts for 10-15% of all human breast cancers and generally displays a poorer prognosis than ER-positive breast cancer.

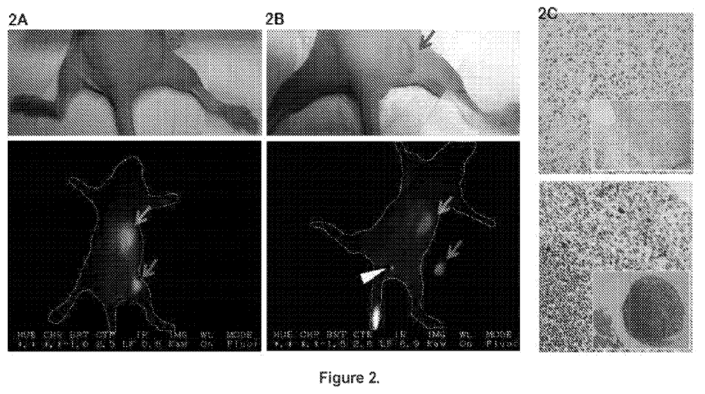

Referring to FIG. 2, Real-time imaging with ICG-p28 in xenograft mouse models is demonstrated. MDA-MB-231 (FIG. 2A) or IOWA-IT (FIG. 2B) breast cancer cells were injected s.c. into mice. Once the tumors (<10 mm) developed, 0.5 mg/kg ICG-p28 was injected i.v. into the mice. Twenty-four hours post-ICG-p28 injection, the mice were imaged with the PDE system during tumor removal. White light photographs (upper) and snapshot NIR PDE images are shown (lower; dorsal view, greyscale at 800 nm). The liver (green arrows) showed relatively high signal intensity from the excretion of ICG. Red arrows: tumors. FIG. 2C, H&E-stained sections of the primary tumor (upper) and a sciatic LN (lower, yellow arrowhead in b) located at the superficial gluteal muscle, which was confirmed to be tumor positive (magnification 200×(inset 40×)).

Athymic mice xenografted with MDA-MB-231 (TNBC) (FIG. 2A) or IOWA-IT (ER+, PR−, Her2−) human breast cancer cells (FIG. 2B) s.c. received a single injection of ICG-p28 i.v. at 0.5 mg/kg (˜125 nmole/kg). Twenty-four hours after ICG-p28 injection, we observed the preferential uptake of ICG-p28 by tumors and clear contrast between the tumor and the surrounding tissue (FIG. 2A-B), suggesting that our approach can be applied to broad types of breast cancer independent of receptor expression patterns. Notably, during tumor resection, intraoperative NIR imaging revealed the preferential uptake of ICG-p28 concentrated over a period of time in a small (≤1 mm) sciatic lymph node (LN) (FIG. 2B). Haematoxylin and eosin (H&E)-stained sections confirmed the presence of cancer cells within the LN (FIG. 2C), suggesting that ICG-p28 can identify other cancer foci in the breast parenchyma.

### Example 2: ICG-p28-Based Assessment of the Tumor Margin in an MDA-MB-231-iRFP Tumor-Bearing Model

To determine whether ICG-p28 can precisely delineate tumor margins, we created an MDA-MB-231-iRFP (TNBC) cell line that stably expresses the NIR fluorescence protein iRFP as a tumor marker. This provides an excellent experimental model to assess the accuracy of tumor margin detection according to a real-time comparison of the fluorescence between iRFP and ICG-p28.

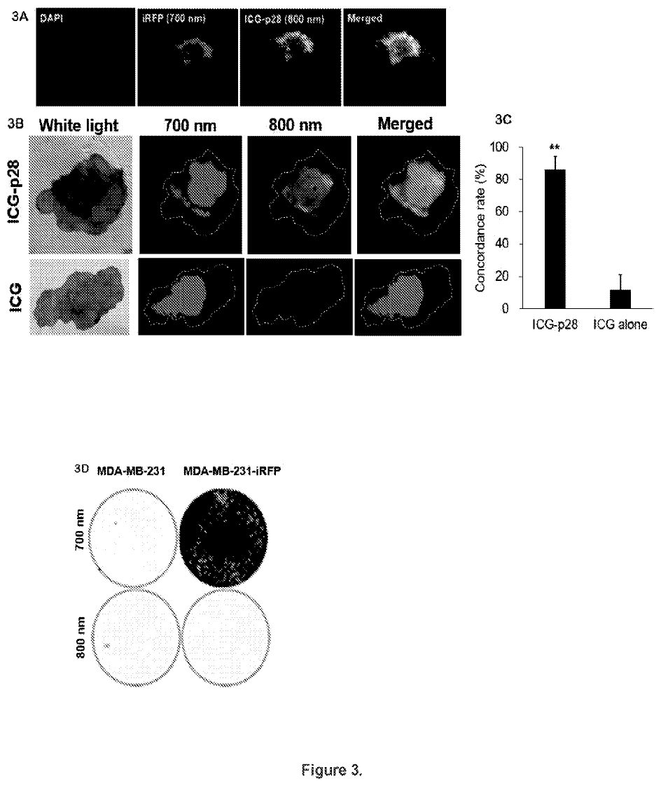

Referring now to FIG. 3A, ICG-p28-treated MDA-MB-231-iRFP cells were fixed in paraformaldehyde, and images were acquired under a confocal microscope. Red: tumor marker iRFP (700 nm); green: ICG-p28 (800 nm); blue: DAPI (nucleus); yellow: red+green. In FIG. 3B, Athymic mice bearing MDA-MB-231-iRFP tumors were injected i.v. with either ICG-p28 or ICG alone at 0.5 mg/kg. Twenty-four hours after injection, representative ex vivo images of the tumour and surrounding soft tissues for the ICG-p28 group (top row) and the ICG alone group (bottom row) were taken with an Odyssey scanner. The dotted line in the images indicates the outer edge of the soft tissue. iRFP (red: 700 nm); ICG-p28 or ICG (green: 800 nm); and colocalization (yellow-orange). In FIG. 3C, Concordance rate of the fluorescent signal in each group [the ICG-p28 group (n=10) and the ICG alone group (n=5)]; **: P<0.01. The results are presented as the mean±SD.

The 700 nm channel refs suitable for detecting tumors generated by these cells in vivo, as it can be used to monitor iRFP-specific fluorescence in cancer cells without affecting the 800 nm fluorescence and thus the signal from ICG-p28. Confocal (FIG. 3A) and LI-COR Odyssey (FIG. 3D) images of MDA-MB-231-iRFP cells showed that the intracellular expression of the tumor marker iRFP allowed the accurate determination of tumor margins. ICG-p28 or ICG alone at 0.5 mg/kg was injected i.v. into athymic mice inoculated with MDA-MB-231-iRFP cells; 24 hr later, the xenografted tumors and surrounding tissues were removed. The dotted lines in FIG. 3B indicate the outer edges of the soft tissues. Ex vivo images showed good overlap between the NIR signals (merged) from iRFP (red: 700 nm) and ICG-p28 (green: 800 nm) (FIG. 3B). Furthermore, similar to the results presented in FIGS. 1 and 2, ICG alone exhibited poor tumor retention (FIG. 3B). The concordance rates of 700 and 800 nm NIR signals in the ICG-p28 and ICG alone groups were 86% and 12%, respectively (FIG. 3C). These results indicate that ICG-p28 can preferentially localize at tumor sites and enhance the stability of ICG due to p28 conjugation.

### Example 3: Intraoperative Imaging of Orthotopic Xenografts

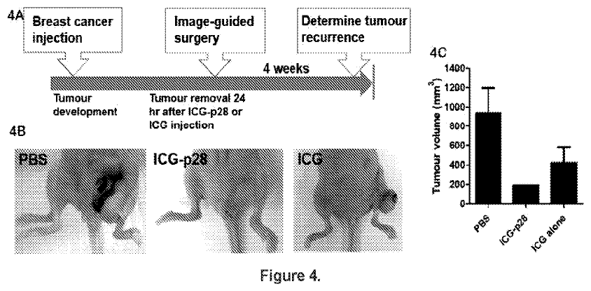

To evaluate whether real-time image-guided surgery can reduce tumor recurrence, orthotopic MFP xenograft tumor models were used. Referring to FIG. 4, Intraoperative imaging of orthotopic xenograft models is shown. In FIG. 4A, A schematic diagram of the intraoperative imaging performed during tumor resection in human breast cancer xenograft tumor models. Human breast cancer cells (MDA-MB-231 or IOWA-IT) were inoculated into the fourth abdominal fat pad via s.c. injection at the base of the nipple. When the mice developed a tumor, they were injected with PBS, ICG-p28 or ICG at a concentration of 0.5 mg/kg. Twenty-four hours after injection, the tumors were resected while being visualized with the PDE imaging unit; the exception was the PBS group, whose tumors were identified only with palpation/direct visualization (no imaging guidance). After tumor resection, the mice were monitored for four additional weeks to detect any tumor recurrence. In FIG. 4B, representative images of tumor resection sites at the end of the study. In FIG. 4C, average volumes of the recurrent tumors (four weeks after tumor resection) in each group. The results are presented as the mean±SD.

Athymic mice bearing TNBC MDA-MB-231 or ER-positive IOWA-IT tumors were treated with PBS, ICG alone or ICG-p28 (0.5 mg/kg, i.v.), and NIR fluorescence was monitored with the PDE system during tumor resection surgery, in which tumors and surrounding tissues with a 2-mm safe margin were removed. During tumor removal, no apparent adverse events related to the administration of ICG or ICG-p28 were observed, and there were no other complications during the procedures. For the control mice treated with PBS, only palpation and direct visualization without fluorescence imaging were used for margin determination. After tumor resection, tumor recurrence resulting from remnant positive margins at the resection sites was evaluated (FIG. 4A). Four weeks after tumor resection, recurrence was observed at the surgical sites in 31.2% ( 5/16) of mice in the PBS group and 25.0% ( 4/16) of mice in the ICG alone group. In sharp contrast, one of sixteen mice (6.3%) in the ICG-p28 group demonstrated tumor recurrence at the resection site. Representative images of the surgical sites in each group are shown (FIG. 4B). The average volumes of the recurrent tumors were 944, 197, and 423 mm3 in the PBS, ICG-p28, and ICG groups, respectively (FIG. 4C). Collectively, these findings show that by aiding in the precise identification of the tumor margins, ICG-p28 improved surgical outcomes by reducing both the incidence of tumor recurrence and the volume of recurring tumors.

### Example 4: Image-Guided Tumor Resection in a PDX Tumor Model

PDX models are widely used in cancer research because of their distinct advantages in preserving the biological characteristics of the original human tumor. Given the reduced tumor recurrence observed in the orthotopic xenograft models treated with ICG-p28-guided resection (FIG. 4C), we utilized a PDX model and a primary TNBC fragment (stage IV) to further evaluate ICG-p28 in a more clinically relevant model. In addition, to investigate whether this result was due to the specific motifs of p28, we included additional control peptides conjugated to ICG. Similar to p28, a noncationic CPP derived from annexins, named AA3H, was previously isolated and shown to penetrate cancer cells. The other peptide was retro-inverso p28 (RI p28). RI modification is often used as an approach to generate a peptidomimetic, which represents the isomer of the parent peptide (p28) in which the direction of the sequence is reversed and the chirality of each amino acid residue is inverted.

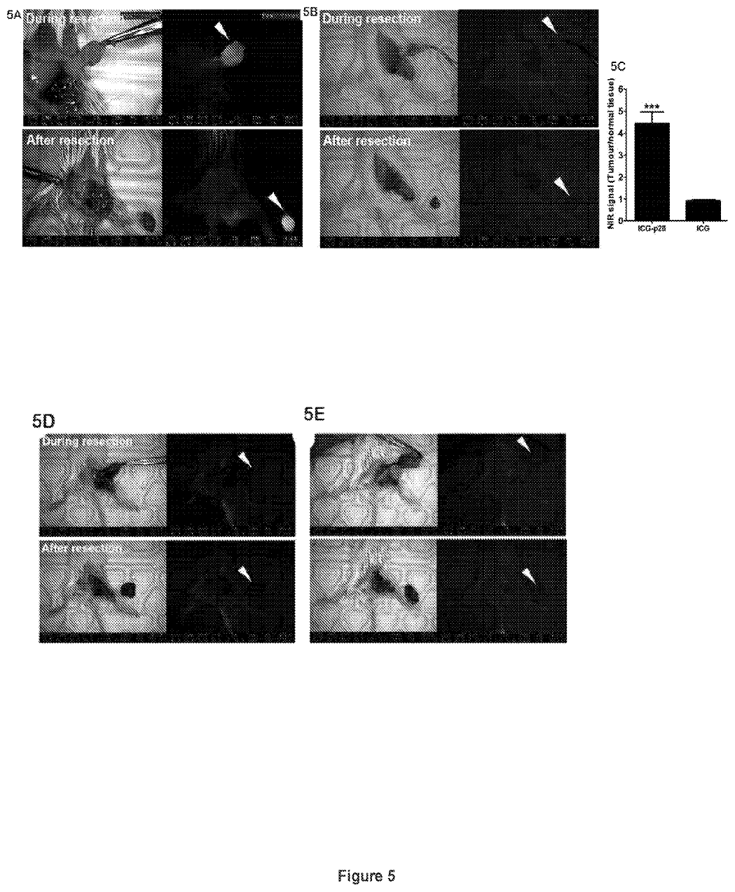

In use then, as demonstrated in FIG. 5, image-guided surgery of human breast cancer PDX models occurs. TNBC PDX cells were inoculated into the fourth abdominal fat pad. Twenty-four hours after NIR agent injection (FIG. 5A, ICG-p28, FIG. 5B, ICG alone), the tumors were resected under visualization with the PDE imaging unit. White light photographs (left) and snapshot NIR PDE images are shown (right; dorsal view, greyscale at 800 nm). Yellow arrowhead: tumor. In FIG. 5C, the signal-to-noise ratio in PDX-bearing mice was recorded with the PDE unit and quantitatively analyzed (N=8 per group). ***: P<0.001. The results are presented as the mean±SD.

Under anesthesia, tumors and surrounding mammary tissues with a 2-mm safe margin were removed under real-time guidance with PDE imaging of mice injected with ICG-p28 (FIG. 5A), ICG alone (FIG. 5B) or a control agent (PBS, ICG-RI-p28 FIG. 5D or ICG-AA3H; FIG. 5E). ICG-p28 provided significantly greater contrast enhancement and a more readily identified tumor than the control agents. These results show that the tumor-targeting ability of ICG-p28 is due to the specific motifs of p28.

### Example 5: Detection of Residual Cancer Cells in the Tumor Margin by Alu Sequencing

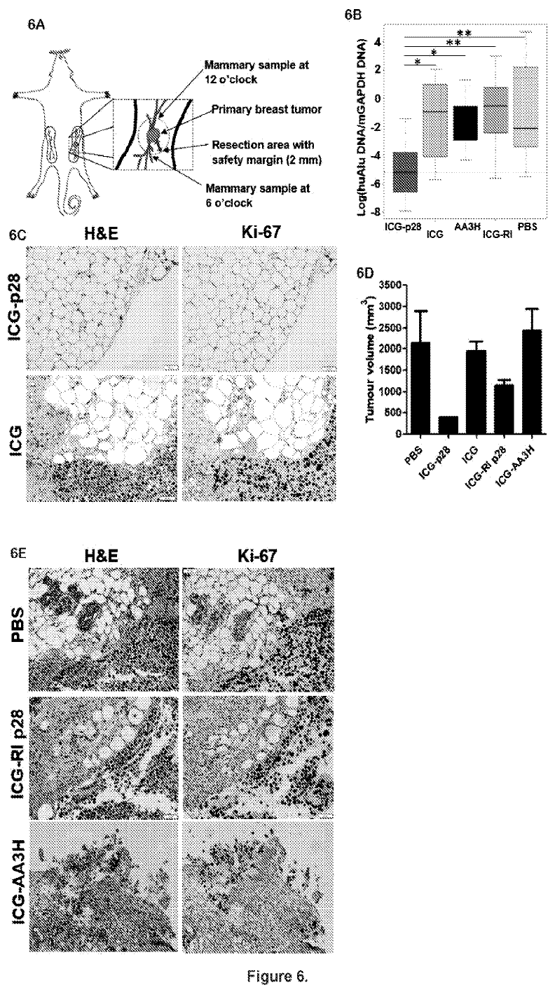

Referring now to FIG. 6, Detection of residual cancer cells in tumor margins is described. FIG. 6A refers to a schematic diagram of the sites of biopsy collection from NSG mice bearing PDX tumors. FIG. 6B demonstrates genomic DNA was extracted from each specimen and subjected to PCR assays. The box plot shows the values of log (huAlu DNA/mGAPDH DNA), calculated using standardized amounts of huAlu DNA and mGAPDH DNA among the agents. *: P<0.05, **: P<0.005. Results are presented as the mean±SD. In FIG. 6C, histological analysis of surgical specimens is shown. Representative images (200×) of H&E staining and staining for the proliferative marker Ki-67 to confirm the presence of negative margins in specimens obtained from the ICG-p28 group (top). The specimen obtained from the ICG group (bottom) shows the presence of malignant cells. In FIG. 6D, four weeks after surgical resection, the tumor volumes in each group were determined. FIG. 6E provides representative images (200×) of H&E staining and staining for the proliferative marker Ki-67 confirmed the presence of positive margins in specimens obtained from the indicated groups.

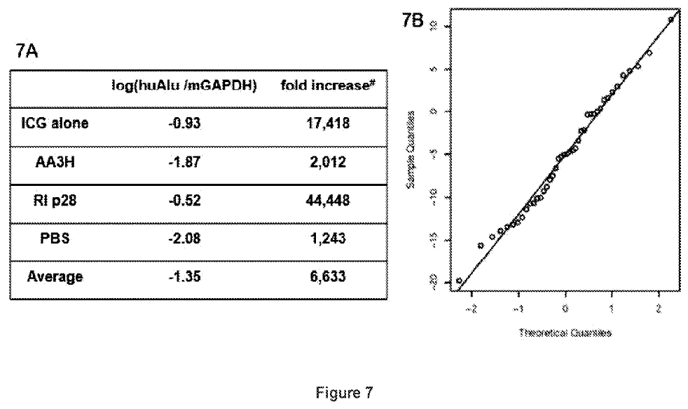

To quantitatively determine the efficacy of margin identification by our imaging approach, PCR-based assays were used to detect residual human cancer cells in the PDX models described above (FIG. 5). During image-guided surgery, tumors with a 2-mm safety margin and the surrounding mammary tissues at the 6 and 12 o'clock positions were removed in real time under guidance of the PDE imaging system (FIG. 6A). These samples were collected to undergo huAlu PCR as well as traditional histological analyses. In the quantitative analysis using huAlu and the housekeeping gene mGAPDH, image-guided surgery with ICG-p28 showed significantly fewer residual cancer cells in the surrounding tissues [log (huAlu DNA/mGAPDH DNA)=−5.2] than surgery with the other agents [ICG alone, ICG-AA3H, ICG-RI p28 or PBS: −0.9, −1.9, −0.5 and −2.1, respectively] (FIG. 6B, FIG. 7).

Referring specifically to FIG. 7, the results of a normality test for huAlu/mGAPDH values are shown. In FIG. 7A, based on the data shown in FIG. 6B, the fold increase over residual tumor DNA in the surrounding tissue from the ICG-p28 group was calculated (#). Image-guided surgery with ICG-p28 resulted in a 6.6×10³-fold average decrease in residual normalized tumor DNA at the margin sites relative to surgery with a control agent. In FIG. 7B, a normality test was performed to evaluate the normality of the distribution of huAlu/mGAPDH values, which were log transformed using the formula Y=log (huAlu/mGAPDH). The QQ plot shows a linear relationship, indicating that the transformed data could be reasonably treated as normally distributed.

This finding indicated that image-guided surgery with ICG-p28 resulted in a 6.6×10³-fold average reduction in residual normalized tumor DNA at the margin sites relative to surgery with the controls and that ICG-p28 precisely distinguished tumor margins better than the other agents. The coefficient of variation (CV) also indicated that ICG-p28 was superior to the other peptides/agents as an intraoperative probe, as it demonstrated a relatively small dispersion shown in Table 1.

TABLE 1
Coefficient of variation from the quantitative
analysis of tumor margins:

| | Coefficient of |
|---|---|
| Probe | Variation |
| ICG-p28 | 8.88 |
| ICG alone | 82.0 |
| AA3H | 5.07 |
| ICG-RI p28 | 32.4 |
| PBS | 902 |

We calculated the coefficient of variation (CV), which indicates variability, as defined by the standard deviation, relative to the mean. For log-normal data, the calculation formula is CV=\sqrt{exp(s²)−1}, where s² is the sample variance. The results clearly show that among all probes, ICG-p28 and AA3H had good CV values (relatively small dispersion). This finding strongly supports the stability of the new agent, ICG-p28.

We next performed traditional histological analyses with H&E and proliferative marker Ki67 staining on the set of tumor margin samples that were used for the Alu PCR assays. The results of these tests revealed that image-guided surgery with ICG-p28 could result in a clear negative margin at the edges of mammary tissues (FIG. 6C). In the ICG-p28 group, one of 15 mammary tissue samples showed atypical hyperplasia, while the positive margin rates of the PBS, ICG alone, ICG-RI p28 and ICG-AA3H groups were 19, 19, 31 and 38%, respectively (FIGS. 6C, 6E). The histological data supported the results obtained from the Alu PCR assay, indicating that image-guided surgery with ICG-p28 improved the ability to precisely identify tumor margins.

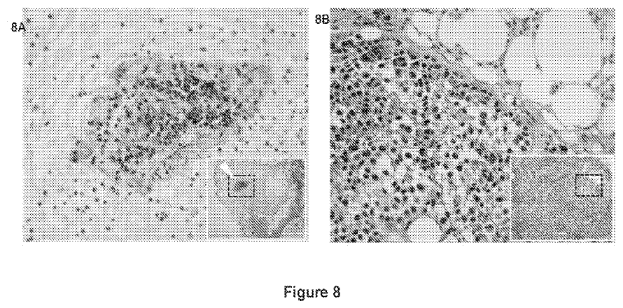

As the tumor recurrence rate is a clinically significant indicator for positive margins, we determined the tumor recurrence rates in the PDX tumor model. Four weeks after image-guided surgery, the tumor recurrence rates for the surgical site were 25.0%, 50.0%, 37.5%, and 25.0% in the PBS, ICG alone, ICG-RI p28 and ICG-AA3H groups, respectively. In the ICG-p28 group, one of eight mice that had atypical hyperplasia during tumor resection demonstrated recurrence (FIG. 8). The average volumes of the recurrent tumors were 2,151, 1,952, 403, 1,152, and 2,447 mm³ in the PBS, ICG alone, ICG-p28, ICG-RI p28, and ICG-AA3H groups, respectively (FIG. 6D). The average volumes of the recurrent tumors in the ICG-p28 group were the smallest among all groups tested. ICG-p28 provided greater contrast enhancement and a more readily identified tumor than the control agents (FIG. 5C), which provided low contrast and made it difficult to remove the tumors completely. These findings suggest that image-guided surgery with ICG-p28 can precisely identify tumor margins, resulting in reduced tumor recurrence.

We found that the tumor-specific signal was optimal at an ICG-p28 concentration of 0.2-0.5 mg/kg within a time scale (24 hr) practical for clinical use. With the imaging and dosing parameters, ICG-p28 provided a significant contrast (TBR˜4-fold) in multiple breast cancer animal models. It is generally accepted that a TBR>3.0-fold is sufficient to provide adequate intraoperative contrast in studies on image-guided surgery and is essential for successful clinical translation. In contrast, ICG alone was excreted rapidly, as expected, and the tumor site was not distinguishable (TBR˜1.1) on the associated NIR images. This observation also confirmed that the preferential uptake of the new compound was due to the presence of the p28 motif, which lead to longer retention at the tumor site and precise contrast imaging between the tumor and surrounding tissue. Although the molecular weight, pI, charge, hydrophobicity, and amino acid composition of RI p28 were exactly the same as those of p28, the overall entry of RI p28 was significantly decreased, and it did not show preferential entry (FIG. 6). This finding suggests that the preferential entry of p28 is amino acid sequence-specific/stereochemically specific. Currently, various types of NIR imaging probes are used for different cancers, such as glioma and lung, pancreatic, colorectal, prostate, and breast cancers. For the latter, a series of investigational fluorescent agents, such as bevacizumab-IRDye800 (vascular endothelial growth factor A), EC17 fluorescent dye (folate-receptor alpha), the cathepsin activatable fluorescent agent LUM015 (a cathepsin protease), and tozuleristide (a matrix metalloprotease), are being validated for use in image-guided surgery; the majority of these imaging agents, however, are preclinical-stage NIR dyes and use an antibody for their targeted delivery (e.g., an anti-Her2 antibody). In comparison, the use of p28 has several advantages: i) unlike that of antibodies, the chemical conjugation of p28 to ICG does not alter ICG fluorescence (FIG. 1), ii) p28 can target a wide range of breast cancers independent of the receptor status (FIGS. 2, 5, 6), and iii) ICG-p28 is composed of the clinically non-toxic tumor-targeting p28 peptide with the FDA-approved NIR ICG dye.

Image-guided surgery with ICG-p28 in the PDX model resulted in tumor recurrence in one of eight mice (FIG. 6D). However, the Alu PCR assay and histological analyses did not reveal residual tumors in the MFP specimen obtained at either the 6 o'clock or the 12 o'clock position. There are two possible reasons for the development of this phenotype. One possibility is that cancer cells were not captured by histological analyses. Although the specimen was determined to be tumor negative by histology and the Alu analyses, it may have contained a small number of tumor cells that were otherwise missed, indicating a possible microinvasive breast tumor measuring less than 1 mm in size. Lesions of this size would not be visible without a microscope and would have been left unresected in the tumor bed. If this is the case, additional approaches (e.g., image-guided surgery with ICG-p28 in combination with other therapeutic approaches such as radiation or chemotherapy) need to be further considered to manage these microlesions. Another possibility is that the atypical hyperplasia that was captured in the histological section (FIG. 8) might have developed into breast cancer during the 4-week observation period. Although atypical hyperplasia is not cancer, it can transition into invasive/noninvasive breast cancer, possibly due to the characteristics of the PDX models. Nevertheless, given that only one mouse in the ICG-p28 group demonstrated tumor recurrence and that the volume of the recurred tumor was smaller than that of the tumors from the mice in the other groups, ICG-p28-guided surgery appears to assist well in precisely identifying tumor margins and to significantly reduce tumor recurrence rates. Overall, image-guided surgery with ICG-p28 in multiple subtypes of breast cancer (FIGS. 4, 6) resulted in an average recurrent tumor volume of 300 mm³, whereas surgery with ICG alone or without interoperative imaging (PBS) resulted in average recurrent tumor volumes of 1,188 mm³ (P=0.017) and 1,289 mm³ (P=0.032), respectively. In addition, the tumor recurrence rate for the ICG-p28 groups was 8% (92% specificity, N= 2/24), while the tumor recurrence rates for the ICG and PBS groups were 33% (N= 8/24) and 29% (N= 7/24), respectively. These results in multiple breast cancer animal models suggest that ICG-p28 improved surgical outcomes by reducing both the incidence of tumor recurrence and the volume of recurrent tumors through clear margin identification. Thus, the versatility of ICG-p28 can potentially reduce the risks of recurrence and reoperation for a broad type of breast cancer independent of the receptor expression status, minimize damage to healthy tissues and healthcare costs, improve postoperative quality of life, and increase patient survival rates.

Many imaging modalities, such as MRI, X-ray, CT, PET and ultrasound, have considerable roles in preoperative staging and intraoperative planning, while fluorescence imaging can be used during intraoperative surgical inspection and practice due to its superior resolution and sensitivity. The precise identification of tumor margins is the major objective for effective surgical treatment of breast cancer as well as many other types of solid tumors. Inadequate positive margin identification frequently occurs in ˜5% of resections of lung and kidney tumors, in ˜20% of resections of breast, prostate and rectal cancer and in up to 40-60% of resections of vulvar and oral cancer. Our real-time intraoperative imaging approach has the potential to positively impact the surgical treatment outcomes (e.g., morbidity, quality of life and costs) of many types of solid tumors.

### Methods

### Peptide Synthesis and ICG Conjugation

Peptides, p28, retro-inverso p28 (RI p28), and AA3H 22 were synthesized by CS Bio, Inc. at >95% purity and mass balance. Peptides were dissolved in PBS buffer at pH 7 and reacted with ICG-maleimide in anhydrous DMSO for 3 hr at room temperature in the dark. The reactions were dialyzed (M.W. 2,000 cutoff, Pierce) against PBS at 4° C. for 24 hr and purified by filtration over Sephadex G-25 (GE Healthcare). The final products were identified by HPLC and MALDI mass spectroscopy.

### ICG-p28 Characterization

The PDE Neo® (Photodynamic Eye; Mitaka USA, UT) is an FDA 510(k)-cleared NIR camera composed of a charge-coupled device (CCD) camera unit that detects an 800 nm NIR signal. In general, the PDE unit (camera connected to a small control unit and LCD monitor) has been used for NIR imaging clinical applications with ICG for identifying hepatic segments and bile ducts. ICG and ICG-p28 in 20% intralipid emulsion (Sigma; St. Louis, MO), which reportedly mimics the light scattering and absorption properties of tissue, were recorded by the PDE camera unit. Various concentrations of ICG-p28 (0.2 pM to 20 μM) and ICG were monitored at 3 mW/cm² excitation.

### Construction of Breast Tissue-Mimicking Phantoms

Optical technology at the NIR wavelength for tissue measurement is advantageous because it can avoid the influence of autofluorescence and allow measurements of relatively deep tissues. To determine the depth detection limit, tumor-simulating inclusions containing ICG-p28 were incorporated into tissue phantoms at predefined locations and imaged with the PDE unit. A previously published procedure for making tissue phantoms was followed with modifications, as illustrated in FIG. 1E. Briefly, 10% gelatin powder (Spectrum; New Brunswick, NJ) was dissolved in 50 mM Tris buffer containing 150 mM NaCl at pH 7.4. The gelatin was dissolved by stirring at 50° C., followed by cooling to 37° C. Bovine hemoglobin and intralipid (20%; Sigma) were then added. To determine the maximal penetration depth of the NIR fluorescence signal, an inclusion containing 200 nM ICG-p28 was positioned in the phantom tissue. Subsequently, the phantom tissue was excised in 2 mm layers at a time towards the inclusion. At all depths, real-time imaging was performed with an intraoperative PDE neo system. The distance between the PDE camera and the phantom was fixed at 200 mm. The intensity of the images acquired using the PDE neo system was rated quantitatively. The mean±SD values were calculated.

### Cell Cultures

The human breast cancer cell lines MDA-MB-231 and IOWA-IT were obtained from American Type Culture Collection (Manassas, VA). MDA-MB-231-iRFP cells were established and characterized in our laboratory. MDA-MB-231 cells were transfected with the piRFP plasmid with Lipofectamine 2000 (Invitrogen). Transfectants stably expressing the iRFP gene were selected in the presence of puromycin at 0.5 μg/ml. They were cultured in MEME medium supplemented with 10% heat-inactivated fetal bovine serum at 37° C. in a humidified incubator with 5% CO₂. Single-generation patient-derived xenograft (PDX) cells of triple-negative breast cancer cells (TM00090) were obtained from Jackson Laboratory.

### Animal Models

All animal work was performed under a protocol approved by the University of Illinois at Chicago. Tumor models were established by subcutaneously (s.c.) implanting 1.0×10⁶ MDA-MB-231, IOWA-IT or MDA-MB-231-iRFP cells into the right back (kinetic analysis) and mammary fat pads (MFPs; qualitative margin analysis) of 4- to 5-week-old female athymic mice (Jackson Laboratory). For additional quantitative margin analysis, tumor models were established by implanting triple-negative PDX cells (stage IV) in the MFPs of 4- to 5-week-old female NOD/Scid/IL2Rγ^{null} (NSG) mice (Jackson Laboratory).

### In Vivo Kinetic Analysis of ICG-p28

MDA-MB-231 cells were injected s.c. into 4- to 5-week-old female athymic mice. ICG-p28 or ICG alone was injected intravenously (i.v.) (tail vein) at 0.2, 0.5, or 1 mg/kg-b.w. Animals were imaged using an Odyssey scanner (LI-COR Biosciences) at 6-72 hr post i.v. administration. The tumor background ratio (TBR) was determined using ImageJ software (National Institutes of Health), where a region of interest (ROI) was drawn over the whole tumor and a background ROI was drawn over adjacent tissue. An ROI was manually generated around each tumor (800 nm channel).

### Tumor Margin Assessment

MDA-MB-231-iRFP cells (1.0×10⁶ cells in 0.1 ml PBS) were inoculated s.c. into the right back of mice. Once mice developed tumors (˜200 mm³), they were injected i.v. with ICG-p28 or ICG at 0.5 mg/kg-b.w. Twenty-four hours after injection, tumors and the surrounding tissue were resected. Specimens were imaged ex vivo with an Odyssey scanner with the 700 nm channel for iRFP and the 800 nm channel for ICG-p28 or ICG alone. The area measurements were performed using ImageJ. The mean±SD values were calculated.

### Quantitative Assessment of ICG-p28 for Tumor Margin Determination and Tumor Recurrence Follow-Up

Once NSG mice developed PDX tumors of similar sizes (200-300 mm³), the mice were randomly divided into five groups: ICG-p28, ICG alone, ICG-AA3H peptide, ICG-RI p28, and PBS. Each agent was injected i.v. (tail vein) with the corresponding compound at the equivalent of ICG-p28 0.5 mg/kg-b.w. Twenty-four hours after injection, all surgical procedures were started under sterile conditions. Following anesthesia administration, the PDE imaging system was used to monitor NIR fluorescence throughout the entire surgical procedure. Tumors with a 2-mm safety margin and the surrounding mammary tissues at the 6 and 12 o'clock positions were removed in real time under guidance of the PDE imaging system. For the PBS group, malignant tissue identified via visual and tactile cues was resected under white-light illumination, while only tissue demonstrating contrast enhancement was resected in the ICG, ICG-p28, ICG-AA3H, and ICG-RI p28 groups; images of the fluorescence signal were displayed on an adjacent monitor, and all ICG-positive tissue foci (suspected as tumor tissues) were excised. Following tumor excision, the skin incisions were closed using a nylon suture, and the mice were returned to their cages. After tumor resection, the mice were kept in their cages for an additional four weeks and monitored for tumor recurrence. Tumor recurrence was monitored by palpitation of the surgical area. Four weeks after surgery, the mice were evaluated to determine whether any of the tumors had recurred due to remnant positive margins at the resection site.

### Detection of Residual Cancer Cells in the Tumor Margin by Alu Sequencing

A quantitative analysis using human-specific Alu sequence (huAlu) was performed on samples collected from the margins. Since Alu-repeat DNA sequences are specific to human cells, human-derived tumors implanted into mice can be identified by detecting the presence of huAlus. We standardized the quantitative detection of huAlu in resected tumors and surrounding margin tissues according to previously published methods. Human tumor cells in the five groups (ICG-p28, ICG alone, ICG-AA3H, ICG-RI p28, and PBS; 8 mice in each group) were identified based on the quantitative detection of huAlu present in the mice. Genomic DNA was extracted from harvested tissues using the DNeasy Blood & Tissue Kit (Qiagen). To identify human cells in the mouse tissues, primers specific for huAlu (sense: 5′-ACG CCT GTA ATC CCA GCA CTT-3′ (SEQ ID NO: 1); and antisense: 5′-TCG CCC AGG CTG GAG TGC A-3′ (SEQ ID NO: 2) were used to amplify the huAlu repeats present in the extracted genomic DNA by real-time PCR (5 ng genomic DNA, 0.5 μM each primer, and PowerUp SYBR Green Master Mix; Life Technologies, USA). Each reaction was performed in a final volume of 10 μl under the following conditions: polymerase activation at 50° C. for 2 min and 95° C. for 2 min followed by 30 cycles at 95° C. for 30 sec, 63° C. for 30 sec, and 72° C. for 30 sec. A quantitative measure of amplifiable mouse DNA was obtained through amplification of the mouse GAPDH genomic DNA sequence (mGAPDH) with mGAPDH-specific primers (sense: 5′-AGG TCG GTG TGA ACG GAT TTG-3′ (SEQ ID NO: 3); antisense: 5′-GGG GTC GTT GAT GGC AAC A-3′ (SEQ ID NO: 4) using the same PCR conditions described for huAlu. The fluorescence emitted by the reporter dye was detected, and the threshold cycle (Ct) was recorded as a quantitative measure of the amount of PCR product in each sample. The amount of huAlu DNA and mGAPDH DNA in the margins was calculated by comparison with a standard curve, and the calculated amount of huAlu DNA was normalized against the relative quantity of mGAPDH. The mean±SD values were calculated.

### Histological Analysis

Resected tissues were placed in containers prefilled with buffered 3.7% formalin (Anatech) and fixed for 24 hr. Formalin was replaced with 70% ethanol following fixation. Samples were paraffin-embedded, and blocks were cut into 4 μm-thick sections and mounted onto slides. H&E- and Ki-67-stained slides were analyzed by a pathologist blinded to the experimental groups and surgical resection results. The pathologist was responsible for determining a tumor's presence, margin status, and general characteristics of resected tissues.

### Statistical Analysis

Data processing and statistical analysis were performed using GraphPad Prism ver. 8 (GraphPad Software) and R version 4.0.5 (The R Foundation for Statistical Computing). Friedman's test for and the F test were used for recurrent tumor volume data and Alu sequences, respectively.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of intraoperatively identifying tumor cells in a subject, the method comprising:
administering to a subject a detectably labeled p28 probe;
performing surgery to remove the tumor cells from the subject;
wherein the probe is localized to tumor cells; and
wherein, during the surgical procedure, an imaging technique that provides real time images is performed to detect cells labeled by the probe, these cells labeled by the probe representing tumor cells.

2. The method of claim 1, wherein the detectable label of the p28 probe comprises a near-infrared fluorescent molecule.

3. The method of claim 2, wherein the near-infrared fluorescent molecule is indocyanine green.

4. The method of claim 1, wherein the probe is administered to the subject a concentration between about 50 nM and about 250 nM.

5. The method of claim 1, wherein the probe is administered intravenously or subcutaneously.

6. The method of claim 1, wherein the time period between administration of the probe and performing the imaging technique is about 24 hours.

7. The method of claim 1, wherein the imaging technique delineates the margins of a tumor.

8. The method of claim 1, wherein the subject has been diagnosed as having breast cancer.

9. The method of claim 1, wherein the imaging technique comprises a CCD camera unit that detect 800 nm near infra-red (NIR) signals.

10. The method of claim 1, wherein the probe is localized to a tumor that is about 1 mm in size.

11. The method of claim 1, wherein the subject has reduced tumor recurrence after surgical removal of a tumor when compared to subjects that had surgery to remove a tumor without the additional imaging technique during the operative procedure.

12. The method of claim 1, further comprising postoperatively evaluating a subject for remaining tumor cells, the method comprising:
administering to a subject that has had surgery to remove a tumor a detectably labeled p28 probe;
applying to the subject an imaging technique to detect cells labeled by the probe, wherein the imaging technique positively identifies cells expressing a tumor marker, thus identifying any remaining tumor cells.

13. A method of detecting cells expressing a tumor marker, the method comprising exposing the cells to a detectably labeled p28 probe; and
performing an imaging technique to detect the probe;
wherein the probe is co-localized to cells expressing a tumor marker, thereby detecting cells expressing a tumor marker.

14. The method of claim 13, wherein the detectable label of the p28 probe comprises a near-infrared fluorescent molecule.

15. The method of claim 14, wherein the near-infrared fluorescent molecule is indocyanine green.

16. The method of claim 13, wherein the probe demonstrates a detectable fluorescent signal at a concentration between about 0.2 pM and about 20 μM.

17. A composition comprising a detectably labeled p28 probe, the detectable label comprising a near-infrared fluorescent molecule.

18. The composition of claim 17, wherein the near-infrared fluorescent molecule is indocyanine green.
